(19) United States
(12) Patent Application Publication
(10) Pub. No.: US 2023/0321399 A1
(43) Pub. Date: Oct. 12, 2023

(54) ROSES SYSTEM FOR ENDOVASCULAR SURGERY

(71) Applicant: Guido DANIEL, Rende (IT)

(72) Inventors: Guido DANIELI, Rende (IT); Salvatore DE ROSA, Staletti (IT); Pasquale Francesco GRECO, Carolei (IT); Ciro INDOLFI, Catanzaro (IT); Gabriele LAROCCA, Polistena (IT); Massimo MASSETTI, Roma (IT); Giovanni TINELLI, Roma (IT); Yamume TSHOMBA, Pozzuolo Martesana (IT)

(21) Appl. No.: 18/023,774

(22) PCT Filed: Aug. 30, 2021
(86) PCT No.: PCT/IT2021/000042

§ 371 Date: Feb. 28, 2023

(30) Foreign Application Priority Data
Sep. 1, 2020 (IT) 102020000020740
Sep. 1, 2020 (IT) 102020000020746
Oct. 22, 2020 (IT) 102020000024622
Aug. 30, 2021 (IT) 102021000022490
Aug. 30, 2021 (IT) 102021000022493

(51) Int. Cl. A61M 25/01; A61B 34/37

(52) U.S. Cl. CPC A61M 25/0113; A61B 34/37; A61B 2034/303

(57) ABSTRACT

This document illustrates all the new features of the ROSES system (RObotic System for Endovascular Surgery) which Is extended by angioplasty to all possible endovascular applications, starting with the increase in size of the passage hole through the RA to allow not only thrust, of catheters and stents of large diameter, but even the passage of hemostatic valves, and then move on to the description of disposable catheters and guides not for angioplasty, then moving on to the RA handling trolley which also allows the measurement of the forces that the organism of the patient opposes the advancement of the catheters, but even that which meets the guide in the advancement inside the catheter. The curvature control system of a catheter is then illustrated, ending with the definition of a new family of RAs that can control two independent rotations and four further independent parameters.

### STATE OF THE ART

Endovascular surgery finds applications ranging from angioplasty, to the elimination of thrombus and aneurysms (very serious in the brain), up to the implantation of aortic valves by percutaneous route, but it has the enormous defect of requiring the participation of the doctor who acts under fluoroscopy to correctly direct the various catheters. For this, robots have been developed that allow doctors to guide catheters and guides using a console.

However, there are currently two types of systems, the one only for angioplasty (Corindus CorPath, American and Robocath, French) while for the wide-spectrum endovascular there is only the Magellan from Hansen Medical. However, no one is able to extend its applications to the entire field, and the measurement of the opposing force from the patient's body to the penetration of the catheters is difficult when it does not even require the use of special catheters, equipped with a sensor on the tip.

ROSES system, EP18807725.9, instead, born in collaboration with Prof. Indolfi and his team of the University of Magna Graecia of Catanzaro, it has already been positively tested also on the patient in its version for angioplasty, while in parallel the PCT/IT2020/050109 shows the possibility to apply to the same ROSES a simple and efficient method for measuring the forces opposed by the body to the penetration of guides and catheters, But in the last year a lot of work has been done in collaboration with Profs. Thsomba, Tinelli and Massetti of the Gemelli Polyclinic in Rome for the extension of ROSES to the entire endovascular system, eventually filing a further series of Italian patent applications, whose internationalization is precisely the subject of this PCT application. Since to extend ROSES to the entire endovascular system, it was necessary to control the movement of catheters even of large diameter, up to 8 millimeters or more, in order to also allow the percutaneous application of the aortic valves, called TAVI (Transcatheter Aortic Valve Implant), which had already been done manually, it was decided, following a discussion with Prof. Massetti and Thsomba, that it was necessary to significantly widen the central hole of the base gear, as described in the question: “New version of the Robot Actuator (RA) “Wide mouth” of ROSES (RObotic System for Endovascular Surgery) for TAVI and related disposables”, N^{o} 102020000020746, presented on Jan. 9, 2020, which was also the basis for a robotic system to guide the fiberscope during the intubation of a patient, ROSINA (RObotic System for INtubAtion), developed following interviews with the anesthetists Dr. Gallo, Maiarota of the Cosenza and Sorbello Hospital of the University of Catania. Among other things, the development of this has also made it possible to perfect the disposable to which the next question refers: “New simplified disposable for ROSES system”, No. 102021000022490, presented on Aug. 30, 2021.

Since among the requests of Prof. Thsomba there was also that of developing a catheter that had a tip with controllable curvature, the question Catheter with tip with controllable curvature from the ROSES robot console for angioplasty and endovascular surgery was studied and presented, and relative disposable, N^{o} 102020000020740, of Jan. 9, 2020 which also uses a trolley that houses several Robot Actuators together with a special two-lumen catheter, one of which for advancing the guide, while the second, smaller, contains a cable that has notches in correspondence with the small lumen, whereby, when pulled, the curvature of a portion of the catheter changes, coupled to a further disposable which allows the curvature of the catheter to be varied from the console.

Discussing then with Dr. Auteri, endocranial radiologist of the hospital of Cosenza, then the application N^{o} 102021000022493 was born presented on Aug. 30, 2021 which describes a method, applicable to the system described by the PCT/IT2020/050109 to measure the force to opposed the penetration of a guide inside of a catheter, to be integrated into the system for measuring the forces opposed by the body to the advancement of guides and catheters.

Always connected to possible mobile tip catheters and from interviews with Prof. De Rosa of the University of Catanzaro, the question “Robot Actuator with three or more degrees of control internal to the main rotation and also with double rotating system” arose, N⁰ 102020000024622, presented on 22 Oct. 2020, created to be able to control a catheter with independent double curvature, even on opposite sides of the catheter itself, and also to make the rotation of the catheter independent from that of the guide, which is not allowed with application N^{o} 102020000020740 previously cited.

### DESCRIPTION OF THE PREFERRED EMBODIMENT

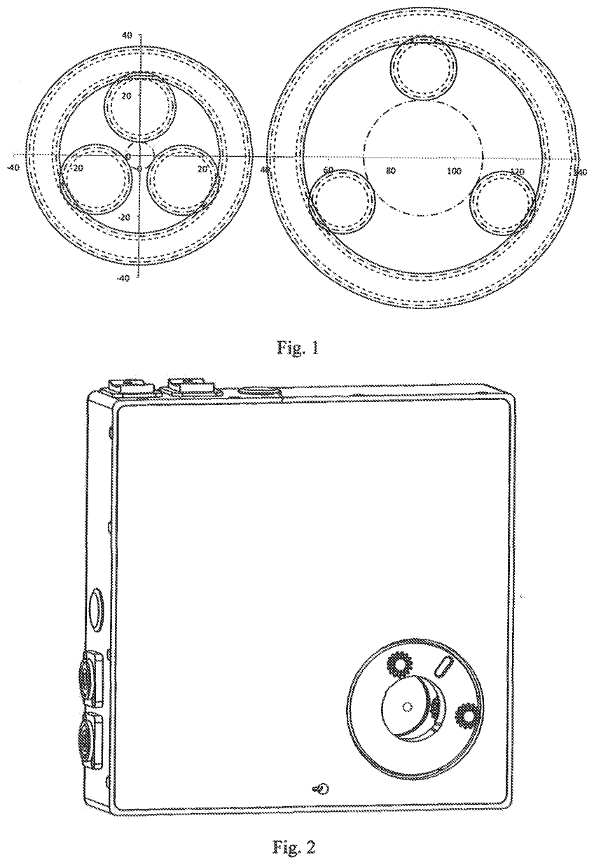

As initially said, to extend to the entire endovascular and try to get up to the implantation of the TAVI, which required not only the passage of catheters of greater diameter, but even the possibility of extracting the catheter and hemostatic valve once the guide was correctly positioned, we have submitted the application 102020000020746, and FIG. 1 shows the comparison between the gearing of the old central mechanism and that of the new one, and it is noticeable how a not very large variation in the number of teeth of the external wheel (from 70 to 97, slightly more than 38%), accompanied by the 47% increase in the number of teeth of the hollow wheel (from 53 to 78) and by the reduction of two teeth of the internal planets of the gearing, which went from 21 to 19 teeth, produced a widening of the through hole 300% of the passage, which passes from 9 to 38 mm in diameter. Subtracting then the two millimeters of the tube intended to separate the gears from the clean part, the increase becomes 386%, passing from 7 to 36 mm in diameter. All this then produced an enlargement of the external dimensions of the complete RA of only 3 cm on the initial approximately 28. While the fixing hole of the push disposables, initially square, has become round with two circular-shaped enlargements on the sides, which serve to allow the univocal positioning of the gear separation tube, which also acts as a sterile rotating connector between the RA cover made of very light plastic and push and rotate disposable for guides and catheters, also uniquely positioned. Obviously, in modifying the toothing, the possible interference between the wheel profiles was taken into account. This produced the new version of the RA, shown in FIG. 2.

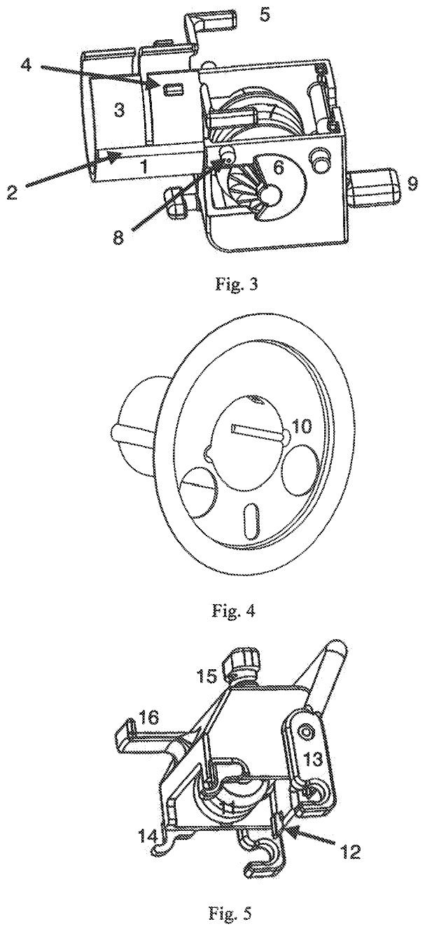

Instead, we take the latest versions of the disposables for large catheters and standard guides from the patent 102021000022490 of Aug. 30, 2021, which, in FIG. 3 shows an image of the component actually made which has the disappearance of the three toothed wheels to transmit the motion to the second. rear friction wheel. So let's see how the two push disposables are made in order to allow the separate fixing of the two components, starting with the lower component. Also in this case we note the presence of a circular attachment open at the top (1) with a diameter of 36 mm that is inserted for 10 mm into the protective tube of the disposable pushers from the internal gears, and has two cylindrical enlargements in the middle (2),), and divided in the upper part into two elements, one more rigid (3) the other which has two teeth (4) which are used to anchor the lower disposable to the tube and are inserted into their housings by pressing on the tabs (5), while the cylindrical enlargements are housed in the lower semicircular grooves (10) of the pipe of FIG. 4, which has changed, with respect to what is illustrated in the Italian patent application number 102020000020746, having lost the two upper cavities present in the aforementioned patent application and which in compensation it must have internally at least 7 non-passing cavities, two of which to house the teeth (4) described for the block of the lower thrust component, a central one for the block of the upper component (16) and from the rear two inlet grooves (17) aligned to those present at the front (10) followed by two grooves which must also in this case receive fixing teeth for a further disposable for the control of the curvature of a catheter, described in the Italian patent application 102020000020740 of Jan. 9, 2020. As can be seen, the lower component takes its motion from the bevel wheel (6), on the left for the catheter, on the right for the guide, which come out of the RA and are not part of the disposable itself, and which in push-fit disposables not for angioplasty will be generally used as an alternative, with the sole exception of the disposable for guides with movable core, described in previous patents, and which will be modified only to adapt to the new RA. The corresponding bevel wheel of the disposable is then fixed on the axis to the single friction wheel (7). Then note the presence of two pins (8) which, as will become clearer later, are used for the attachment and subsequent rotation of the upper component. Finally, the key (9), once turned, anchors the tube and the lower thrust disposable to the first wheel of the gearing, while the two teeth (4) do not allow the deformation of the lower component during the transmission of motion, which caused the bevel gear teeth under stress.

FIG. 5 then shows the upper component, which obviously contains the friction counter-wheel, (11) placed immediately above the axis of the friction wheels of the lower component in the left portion, dedicated to the catheters, but has four major differences compared to this. First of all, the power take-off is missing, then it has two teeth (12) that are inserted inside the lower component and that check the correct positioning of the axes, then there is always a hook (13) that closes the two components by tightening them 1° against each other, and finally, instead of presenting two cylinders at the end of the circular semi-tubing as in the lower component, this semicircular element is completely missing while it has two arms (14) that connect to the two pins of the lower disposable, being also the upper part that faces the front wheel inclined backwards by the same 30° degrees, so that this part of the disposable can be inserted, inclined precisely by 30°, rotated to close the disposable and locked with the locking lever. It should also be noted that the upper friction wheel is placed on a small frame that covers the wheels themselves at the top, while a screw (54) allows to adjust the thrust of the upper wheels on the lower one. Finally (16) is a further tooth that also anchors the upper element to the tube of FIG. 4, increasing the thrust. FIG. 6 shows the two lower and upper disposables assembled, on the left closed in the tube, which in this view also shows the notches and teeth already mentioned (17) to connect the disposable to command the curvature of a special catheter, while on the right without the protective tube and open so as to better show the system assembly.

Obviously, the 0.035″ (approximately 0.9 mm) push disposable guides will differ from what is described above only for the different friction wheels, which do not have the groove like those for catheters, given the smaller diameter and the decidedly structure more rigid than the guides themselves, and above all the grip of the motion, which in this case will be on the right side of the lower element, instead of on the left side.

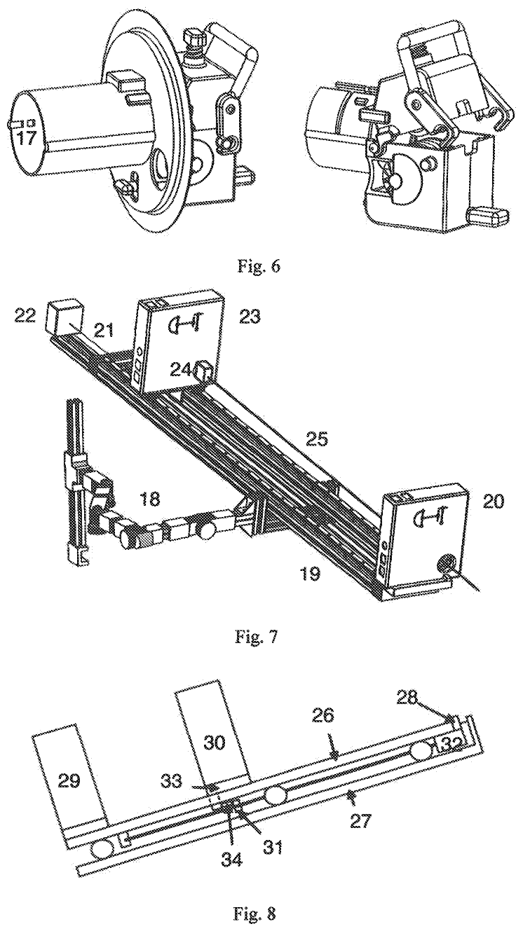

FIG. 7 presents an overview of the trolley configuration for endovascular surgery, contained in the application 102020000020734 submitted on Jan. 9, 2020. This must be fixed to the side of the table, adjustable in height and with the part that holds the RAs composed of two components, a fixed base that can be tilted towards the patient (18) while above this there is a slide (19) which can slide a few centimeters from its lower support. A first RA (20), which we will call proximal, and which usually controls the motion of the catheter, is then fixed on the sled near the patient. The slide is then connected to the fixed lower part with a cable (21) of direction parallel to the slide, kept at a certain distance by a bar fixed to the lower part of the carriage, in turn connected to a force sensor (22). This is also the reason why it is necessary (in addition to being better for the patient's comfort) to tilt the trolley towards the patient so that there is a force of fixed quantity between the sensor, in a fixed position, and the RAs placed on the part, upper part of the carriage, free to slide, albeit with a very limited stroke. And the variation of this force is entirely due to the reaction of the body if the forces of inertia due to the movement of the distal RA are negligible or taken into account.

A worm screw or any other precise method of position control, driven by a step motor, allows moving the distal RA (23) on rails with respect to the proximal, during catheter introduction, so that the relative position of catheter and guide wire is kept constant, enabling the doctor to modify it during their introduction. Practically, during this phase is the catheter that commands the advancement, with the guide wire that may simply advanced or refracted with respect to the catheter. The same cart may also be used with a single RA for angioplasty.

Let's pass to briefly describe the method to measure also the force necessary to push the guide inside the catheter (Italian patent application N^{o} 102021000022493 presented on Aug. 30, 2021) in which FIG. 8 shows a section of the working scheme, presenting the slide (26) placed on a fixed base inclined toward the patient, kept in position by a wire (27) hooked to the base and kept parallel to the slide direction, that coincides also with the direction of penetration of the catheter, where a force sensor (28) is inserted between the wire and either the base or the slide, the first proximal RA, acting on the catheter (29), being fixed on the slide, while the second RA (30), distal, is placed on a plate moving on rails, pushed by a belt or better by a worm screw and relative nut screw (31), actuated by a step motor (32), while between the moving plate (33) and nut screw in placed a second force sensor (34), the allows reading, again by difference between the gravity component acting on the RA and the forces the oppose motion of the RA outside it, the force opposing the guide advancement.

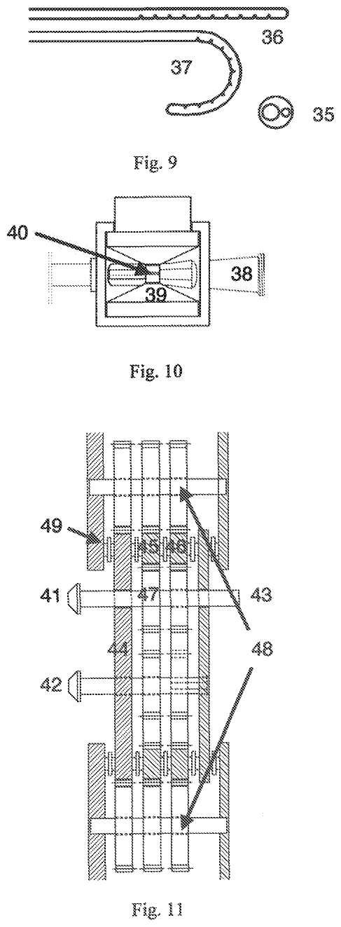

Let's move on to the description of the catheter curvature control system, patent application 102020000020740 of Jan. 9, 2020, starting with how the catheter is made. We therefore consider a tube that has two internal lumens, a larger one for the passage of a 0.035″ guide, and the second small one, for the passage of a cable of about 0.5 mm in diameter, whose section (35) is shown in FIG. 9, on which small wedge-shaped incisions (36) were made on the side of the small lumen. By introducing a small cable in the lumen, locked at the tip, a catheter is obtained that bends from the side of the small lumen (37) by pulling the thread that passes through this lumen. The wire itself remains in the small lumen up to the catheter connector, while the notches can be in a variable position and number depending on the degree of curvature desired.

On the connector side, on the other hand, the connector of this catheter must be present, which must contain a small diameter drum on which the cable must be wound, which must be pulled to cause the catheter tip to bend. FIG. 10 shows the cross section of the catheter connector (38) incorporated in the drum support (39) in which it can be seen how the wire (40) is wound precisely on the drum, which has a very small diameter to increase the accuracy of the control of curvature.

This naturally implies the need for the catheter to be maneuvered by a particular disposable, which must be fixed to a Robot Actuator (RA) that follows the advancement of the catheter. Therefore this RA will need to move at the rate of entry of the catheter into the patient's body. This of course implies the use of the trolley (FIG. 7), on which there are two RAs, one fixed (20) placed near the patient, bearing on the distal side the disposable catheter push (not visible) and the other movable (23), which will have to house, on the rear face, the disposable push of the guide (also not visible), while on the anterior one, on the patient side, the disposable for actuation of the catheter tip (24), represented for simplicity as a box, from which the catheter (25) exits, which also passes through the fixed RA (20).

It should also be noted that while until now the RAs used in ROSES transmitted two rotations only from the back, that is the side from which the push and rotation disposables were introduced, it will be necessary to extend the shaft that in the disposable for angioplasty is used to make advance the catheter, so that it emerges from the patient's side. In this way it will be possible to mount the disposable for pushing and rotating the guide from behind, while from the front a new disposable will be created that will contain both the hemostatic valve and the catheter connector complete with pulling drum of the bending cable, which will be precisely commanded by the Console.

FIG. 11 shows the section of the central rotating element from the RA in correspondence with the shaft which is usually intended solely for the advancement of the catheter, through one of the two bevel wheels (38) and (39), and note the shaft which comes out on the opposite side (40). Note that the various large wheels, of which the first is solid (41) and the other two hollows (42) and (43) which mesh with internal wheels, one of which (44) integral with the wheel pin (38), while the others are idle, are kept in alignment by wheels external to the group (45) of the three central wheels (41), (42) and (43) and separated from each other and from the frame by roller bearings (46).

Note that in this case the two RAs must receive the rotation command at the same time, which must be identical, since the anterior (proximal) one produces the rotation of the catheter, but the same rotation must be performed by the distal RA, which commands the curvature of the tip of the catheter, which implies that the guide will also rotate at the same speed and in the same direction. As for how the disposable for this application must be made, this differs in the meantime by the fact that it must be fixed on the opposite side of the push side of the guide, so it must be completely different from the previous ones, and also in this case centered on the guide disposable gear cover, for example through two internal enlargements in the final part of the gear cover tube of FIG. 4, to allow the exact alignment of the components, while the shaft that protrudes at the front (40), which until now was not required in the RAs, it will simply have to have two opposite flattening so as to be able to transmit the torque necessary to pull the cable that controls the curvature of the catheter. Inside it must then contain a female bushing of the same shape that will carry the rotation beyond the hemostatic valve to end up with a small transmission of toothed wheels, described below, which will control the rotation of the drum, Transmission that can also be disengaged if one then needs to use all the commands on the push disposable placed behind, which could happen if one decides to use this system for angioplasty having used the variable tip catheter to get into position, and obviously does not want the tip of the catheter to change curvature once it has reached the position. However, this would also make it possible to vary the branch of the coronary arteries to be inspected, returning to move the introducer catheter and its tip with variable curvature.

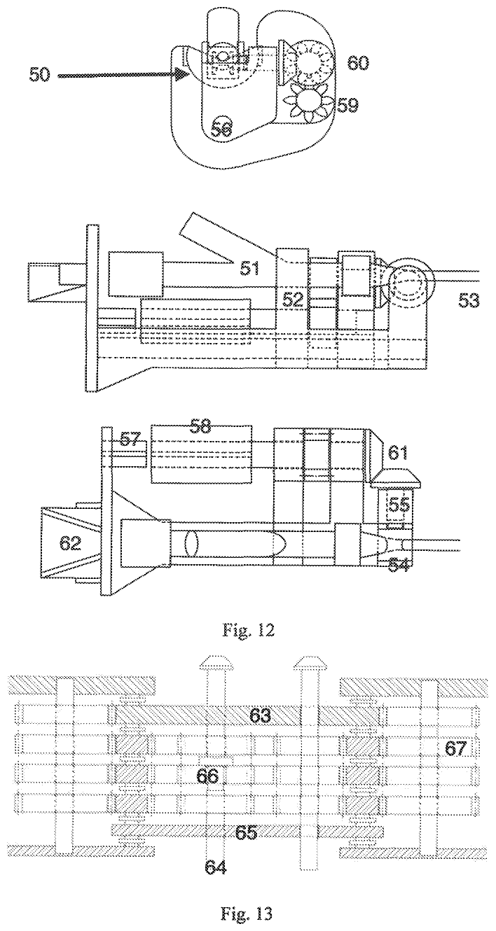

FIG. 12 therefore shows the diagram of the new disposable, in front and side view showing the disposable to be applied to the exit hole of the RA (47) opened from above to be removable without having to remove everything that passes into the RA, as indicated below on which both the haemostatic valve with inlet of the washing liquid (48) are centered, obviously supported by a special support (49) in which it must be inserted, and the catheter (50) under which the drum (51) is schematized which must be turned to operate the tip and the shaft that connects it to the sprockets (52), which is not part of the drum, but must be connected to it. The hole (53) then allows fixing the disposable to the RA, by means of a screw or key. On the side, the shaft (54) coming out from the rear of the RA, represented but not part of this disposable, which attaches and detaches to the transmission through a toothed bushing (55) in order to allow it to be engaged and disengaged, Mounted on this is a toothed wheel with 8 teeth, thrust angle 30° (56), which meshes with an equal wheel placed at the top (57), which then, by means of a pair of bevel wheels (58), rotates the drum which pulls the cable that controls the curvature of the tip. Finally, it should be noted that behind the flat surface, in correspondence with the exit hole from the RA, there is the presence of a semi-funnel (59) which serves to centrally address the guide or what must be introduced into the hemostatic valve.

Conversely, for applications where it is necessary to leave the guide in position by eliminating the catheter, it was decided that the disposable that controls the catheter must have partial and not total coverage of the exit hole, so that, once the guide is brought into position taking advantage of the catheter's curvature control, the disposable that commands it can be removed from underneath, while the catheter with all its actuator drum and the hemostatic valve can be extracted through the hole in the RA, temporarily releasing the disposable push of the guide, so that the guide itself can remain in position, and the new catheter for pushing large stents can be inserted on the tail of the guide, which has remained uncontaminated and therefore sterile until that moment. The only point to check is the size of the hemostatic valve, which must also be small enough to pass through the central hole of the RA, and, if a hemostatic valve small enough is not easily found, just increase the size of the hole a little more. RA, quite simple thing.

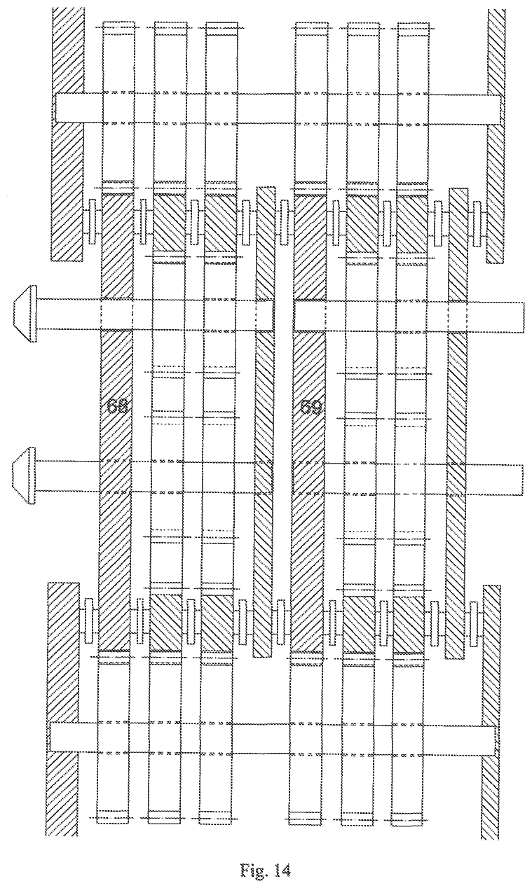

FIG. 13 shows how the addition of, for example, the third degree of control in addition to the main rotation (60) can be achieved by using the second shaft currently used to make the guide advancement control (61) come out of the front wheel, while from the closing plate integral with the front wheel (62) exits the command for the second degree of curvature of the catheter without touching the guide advancement one. As you can see, the shaft is now divided into two sections, one leading to the front, the other, coaxial but separated from the first by a very small roller bearing (63), which carries the command beyond the rear plate (62). Finally, note how the wheel system is kept aligned not only by the roller bearings, but also by a system of free-to-rotate external wheels (64), except for one which is directly connected to the motor, equally spaced angularly.

FIG. 14 instead shows in section the two-carrier system (65) and (66) which allows the independent rotation of catheter and guide, and of a catheter which in the case of the drawing must control a catheter with two degrees of freedom. As you can practically see, there is a simple doubling of the train carrier, which then sends two commands also from the opposite side. Naturally, if only one independent command is needed for bending the catheter, this is achieved simply by eliminating one of the two hollow wheels in the second train carrier.

1. ROSES system (RObotic System for Endovascular Surgery) characterized by the extension to all possible endovascular applications, by increasing the size of the passage hole through the Robot Actuator (RA) to allow not only the thrust of catheters and large stents diameter, but even the passage of the hemostatic valves, using new disposables for catheters also of large diameter and standard guides, divided into upper and lower components to allow separate extraction, and a new trolley for handling the RAs that also allows the measurement of the forces that the patient's body opposes to the advancement of the catheters, but even that which encounters the guide in the advancement inside the catheter then including the control system of the curvature of a catheter, also defining a new family of RA capable to control two independent rotations and four further independent parameters;

2. ROSES System (RObotic System for Endovascular Surgery) as per claim 1 characterized by a new version of the ROSES Actuator Robot in which, thanks to an increase in the number of teeth of the central wheel, a passage hole has been obtained through the RA , protected by a sterile disposable cover, from 7 to 36 mm, thus allowing the insertion of large disposables for the passage of large catheters, while the hole on the first wheel assumes a circular shape with a diameter of 36 mm while previously it was square, also presenting on the sides two rectangular notches for the univocal centering of what must be introduced, also adding the possible exit of the shaft axis which in disposable for angioplasty controls the advancement of the catheter, on the opposite side of the push disposable, or on the side of the patient with shaft facet to transmit torque;

3. ROSES system (RObotic System for Endovascular Surgery) as per claim 1 characterized by the fact that the disposables that separate guides and catheters from the RA mechanisms are divided into a tubular component that completely separates what is contained there from the RA mechanisms, while the push mechanism of guides and catheters is divided into a lower component of the disposable push for catheters, and an upper one that can be extracted separately leaving undisturbed what passes through the hole of the RA, presenting the lower component first of all a circular mouth open at the top (1) with a diameter of 36 mm, about 10 mm long in the protective tube of the disposables pushed by the internal gears, and has in the middle two cylindrical enlargements (2), which fit into the lower semicircular grooves (10) of the tube, which is varied, with respect to what is illustrated in the Italian patent application number 102020000020746, having lost the two upper cavities present in it the aforementioned patent application and which on the other hand must have internally at least 5 non-passing cavities, two of which to house the teeth (4) described below for the blocking of the lower thrust component, a central one for the blocking of the upper component (16), and finally in the rear part two diametrically opposite grooves followed by two non-passing cavities for blocking the disposables which controls the variation of the curvature of the catheter, being also in the upper part of the lower thrust disposable, in addition to the enlargements, present two teeth (4) that penetrate into special cavities inside the tube (10), to allow the entry of which there are two fins (5), pressing which the upper half-tube deforms allowing the teeth to enter their housings, while, as con be seen, the lower component takes the motion from the bevel wheel (6), on the left for the catheter, on the right for the guide, which come out of the RA and are not part of the disposable itself, and which in push disposables not for angioplasty will generally be used alternatively, with the sole exception of the disposable for guides with movable core, described in previous patents, and which will be modified only to adapt to the new RA, while the corresponding bevel wheel of the disposable is then fixed on the axis to the single friction wheel (11), being also present two pins (12) which, as will become clearer later, are used for the attachment and subsequent rotation of the upper component, and finally, the key (9), once turned, anchors the tube and the disposable lower thrust to the first wheel of the gearing, while the two teeth (4) do not allow the deformation of the lower component during the transmission of the motion, which caused the teeth of the bevel gear under stress, while the upper component, characterized by the fact that it has a rhomboid shape when viewed from the side, with a wall facing the RA inclined by about 30° and has two lateral arms (14) suitable for connecting to the pins (8), to act as a pin to initially insert the disposable at an angle of about thirty degrees, then, by rotating, make it adhere to the lower component, being also present, on said upper component, the friction wheel (11) companion of that of the lower component, whose axes must lie exactly on the vertical of the lower ones, in order to block the catheter to allow its advancement, that a tooth (12) suitable for inserting into the component and lower to ensure correct alignment between the components, a double hook (13) that can be operated by hand to permanently fix the two components together, while the upper friction wheels are mounted on a frame covered by a cap on which a screw (15), while a tooth (16) inclined by a certain amount, once the upper component has been positioned, presses the protection tube from the inside, increasing its thrust, since it is possible to replace this protrusion with a tooth that fits into a special tube groove that further compacts the final disposable by increasing the push capacity of the disposable itself;

4. ROSES system (RObotic System for Endovascular Surgery) as per claim 1 characterized by the presence of a trolley containing one or two RA of the ROSES system for angioplasty or endovascular surgery to be fixed to the operating table, adjustable in height and with the part that holds the RA composed of two components, a fixed base that can be tilted towards the patient (18) while above this there is a slide that can slide a few centimeters with respect to its lower support (19). A first RA (20), which we will call proximal, and which usually controls the motion of the catheter, is then fixed on the sled near the patient, being the slide then connected to the fixed lower part with a cable (25) of direction parallel to the slide, kept at a certain distance by a bar fixed to the lower part of the cart, in turn connected to a force sensor (21), while the second RA is fixed to a base moved by a worm screw system or other equivalent system that allows relative movement between the RAs, which is essential during the introduction of the catheters, being a second sensor interposed for example between the nut screw and the platform mobile, which measures the force always in the direction parallel to that of the slide, which realizes the possibility of reading, again by difference between the component of gravity that acts in the direction of the slide, and the forces that oppose outside the pushing Robot , to the advancement of the guide, making it possible to read the force encountered in the advancement of the guide;

5. ROSES system (RObotic System for Endovascular Surgery) as per claim 1 characterized by the presence of a console control system for the curvature of the tip of a catheter composed of a two-lumen catheter (33), one of which for 0.035 mm guides and the other of about 1.5 French characterized by the fact of having the bendable tip (35), presenting the catheter on the tip side of the small wedge-shaped cuts (34) in correspondence with the smallest lumen, being present at the tip inside the small lumen a cable fixed at the tip, which then runs through the entire catheter up to the connector, inside which it is wound on a small radius drum (37), by a slightly modified ROSES RA system to carry the command of the cable (43) on the opposite side to that where the push disposable of the guide is positioned, and from a special new disposable to be mounted on the distal RA (50), on the opposite side to that where the push disposable of the guide is fixed guide, in its own right lta mounted on a mechatronic trolley, which allows you to pull the thread inside the catheter obtaining the desired curvature, the distal R.A. being also modified in order to control the differentiated advancement of the guide with respect to the catheter with a disposable placed on the opposite side of the patient, and from part of the patient, the control of the curvature of the same, using a new disposable to control the curvature of the tip of a catheter from the console characterized by the fact that the disposable itself centers itself by slipping into the mechanical protection tube of the RA which has two opposite protrusions, furthermore presenting a support (52) for the hemostatic valve with inlet of the washing fluid and a bushing (58) which can be easily inserted and removed on the specially splined shaft (57) that protrudes from the RA in order to allow the insertion and disengagement of the transmission, to end with a small cylindrical gear wheel (59) which meshes with a second wheel cylindrical (60) which in turn transmits motion to a pair of bevel gears (61) whose output shaft (55) engages in the drum which activates the bending of the tip, while the catheter connector (36) is blocked into position by securing the catheter to the hemostatic valve and inserting the same into its holder;

6. ROSES system (RObotic System for Endovascular Surgery) as per claim 1 characterized by the presence of a console control system for the curvature of the tip of a catheter which, therefore, uses the three degrees of control of the RA and the motorized trolley, as that the catheter is pushed forward by the proximal RA, the distal RA also advances at the same speed on the catheter while also the rotation of the catheter, commanded on the proximal RA, is repeated identically on the distal RA, so as not to transmit unwanted twists to the catheter, while, if you want to bend the tip, just rotate the head of the joystick that controls the advancement and rotation of the catheter to cause its curvature, which is maintained until a small counter-rotation is given to the head of the joystick, causing the release of the traction of the cable, so as to allow to push the catheter into the desired lumen after having introduced the guides, since the catheter can already be rare only on the side of the small lumen, but allowing the rotation of the disposable to orient the catheter curvature in any direction;

7. ROSES system (RObotic System for Endovascular Surgery) as per claim 1 characterized by the fact that the Robot Actuator with three or more degrees of control on a rotating system, deriving from the European patent application EP18807725.9, consisting of a first toothed wheel of sufficiently large diameter (63) which internally holds a carrier on which pinions are placed which mesh with the internal toothing of a certain number of hollow toothed wheels whose number equals the desired degrees of control in addition to the main rotation, wheels which externally have, for simplicity, but not necessarily, the same toothing of the main wheel, while the various pinions that mesh on each hollow wheel can carry the desired command externally, which can come out both from one end of the carrier and from the opposite side, being possible other pinions which engage on each hollow wheel free to rotate and present only to improve the compactness of the i together;

8. ROSES system (RObotic System for Endovascular Surgery) as per claim 1 characterized by the fact that the Robot Actuator with three or more degrees of control on a double rotating system, deriving from the European patent application EP18807725.9, consisting of two distinct groups of sprockets (68) and (69) and relative hollow wheels with respective meshed pinions, so as to allow the independent rotation of the two sprockets, whose outputs in this case must be on opposite sides.
